(19) United States
(12) Patent Application Publication
(10) Pub. No.: US 2008/0103606 A1
(43) Pub. Date: May 1, 2008

(54) Templated islet cells and small islet cell clusters for diabetes treatment

(76) Inventors: Cory Berkland, Lawrence, KS (US); Lisa A. Stehno-Bittel, Bonner Springs, KS (US); Teruna Siahaan, Lawrence, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/589,063

(22) Filed: Oct. 30, 2006

(51) Int. Cl. A61F 2/02

(52) U.S. Cl. 623/23.72; 424/423; 435/6

(57) ABSTRACT

An implantable biomaterial scaffold having islet cells or small islet cell clusters attached thereto in a multilayer. The cells are derived by enzymatic dispersion and/or calcium depletion of large adult intact islets.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

The present invention generally relates to compositions and processes for creating viable islets cells and small islet clusters attached in a multilayer to a biomaterial scaffold for transplantation.

### DESCRIPTION OF RELATED ART

The rise in cases of diabetes mellitus in the United States has been called an epidemic. Diabetes is the third leading cause of death by disease and rivals heart disease and cancer as a major killer of United States citizens. For unexplained reasons, the occurrence of type 1 diabetes is increasing worldwide, and the age of onset has decreased by three to five years over the past decade so that many children now develop diabetes prior to entering school. The results is that more people with diabetes will spend a larger percentage of their life at risk for developing the chronic complications related to type 1 diabetes. Since the risk for development of most of the chronic complications associated with diabetes is related to glycemic control, significant attention is directed toward novel therapies, such as islet transplantation, to improve glycemic control.

Islet transplants were first attempted in the 1980s. Initial success rates for islet transplantation in humans were disappointing with only 5% of patients receiving transplants achieving partial function. See Sutherland et al., Evolution of kidney, pancreas, and islet transplantation for patients with diabetes at the University of Minnesota, Am. J. Surg. 166: 456-491 (1993). Amid the failures were isolated success stories of individuals achieving prolonged reversal of their diabetes for a 1 to 2 year period, which encouraged researchers to continue this approach to treatment of diabetes. In 2000, islet transplantations were performed successfully on seven patients with diabetes using a suppression regimen that omitted glucocorticoids, now referred to as the Edmonton protocol. See Ridgway et al., Pancreatic islet cell transplantation: progress in the clinical setting, Treat Endocrinol. 2(3):173-189 (2003). Thus, islet transplantation outcomes have improved markedly. See Shapiro et al., Clinical results after islet transplantation, J. Investig. Med. 49(6): 559-562 (2001); Balamurugan et al., Prospective and challenges of islet transplantation for the therapy of autoimmune diabetes, Pancreas 32(3): 231-243 (2006). Yet, regardless of the optimism generated by these results, barriers to the use of islet transplantation as a practical treatment for diabetes still exist, with one of them being the limited number of donor organs considering that most require multiple transplants to achieve insulin independence.

Many factors may have an affect on transplantation success, including the physical characteristics of the islet. About 20 years ago, researchers described in detail the size and shape of islets and determined a method for estimating islet volume. See Bonnevie-Nielsen et al., Pancreatic islet volume distribution: direct measurement in preparations stained by perfusion in situ, Acta Endocrinol. (Copenh) 105(3): 379-84 (1984). For many years, large islets have traditionally been considered desirable by transplant sites for several reasons: (1) the presence of large islets is considered a hallmark of a good pancreatic digestion, since islets can be fragmented by excessive digestion, and (2) volume is used to determine the minimal number of islets needed for transplantation, and because doubling an islet's diameter is equivalent to an eight-fold increase in its volume, large islets make a major contribution to the number of islet equivalents in a preparation.

In recent years, researchers have modeled the transport of oxygen, glucose, and insulin through the islet. See Dulong et al., Contributions of a finite element model for the geometric optimization of an implantable bioartificial pancreas, Artif. Organs 26(7): 583-9 (2002). Limited transport of oxygen can propagate cell death in the core of islets if the rate of oxygen consumption by peripheral cells exceeds the rate of oxygen diffusion into the islet. For example, recent studies indicate that larger islets exhibit increased necrosis when exposed to hypoxic conditions. Indeed, nearly all beta cells died when islet diameter exceeded 100-150 μm. See Giuliana et al., Central necrosis in isolated hypoxic human pancreatic islets: evidence for postisolation ischemia, Cell Transplantation 14: 67-76 (2005); MacGregor et al., Small rat islets are superior to large islets in in vitro function and in transplantation outcomes, Am J Physiol Endocrinol. Metab. 290(5): E771-779 (2006). The resulting oxidative stress can aggravate apoptosis and immune response upon transplantation. See Bottino et al., Response of human islets to isolation stress and the effect of antioxidant treatment, Diabetes 53(10): 2559-68 (2004). Even in cases where cell death has not occurred, a decreased metabolic rate in the islet core is probable.

Retarded transport of glucose and insulin also diminishes the functionality of pancreatic islets. The glucose gradient within an islet causes peripheral cells to contact much higher concentrations of glucose than those contained in the islet core. See Kauri et al., Direct measurement of glucose gradients and mass transport within islets of Langerhans, Biochem Biophys Res Commun 304(2): 371-7 (2003). The shape of this gradient is directly related to the diameter of the islet and the rate of glucose metabolism. Increasing islet diameter increases this diffusional and consumptive barrier in all planes within the islet.

To find another source of insulin-producing beta cells, there have also been efforts to culture beta cells in vitro. These methods have focused on the culturing of beta cells from fetal tissue or deriving such cells from islet-producing stem cells or progenitor cells. See, e.g. Peck et al., U.S. Pat. No. 6,703,017; Brothers, WO 93/00411 (1993); Neilsen, WO 86/01530 (1986); Zayas, EP 0363125 (1990); Bone et al., Microcarriers: A New Approach to Pancreatic Islet Cell Culture, In Vitro Vol. 18, No. 2 February (1982). Unfortunately, such techniques are generally time consuming and require the availability of rare fetal tissue or stem cells as their source and result in a confluent monolayer of cultured beta cells. Thus, there remains a need to create viable islets cells using more efficient, available, and reliable techniques.

In an attempt to overcome the diffusional barrier encountered in the architecture of an large intact islets, various attempts were made by the present inventors to grow multiple layers of islet cells on polymer microspheres for implantation. The microspheres shown in FIG. 1A were engineered to be within the size range of intact islets. By attaching beta cells to the outer surface of the microsphere, it was theorized that there should be little or no cell death due to diffusional barriers. Multiple attempts were made using different culture environments to optimize the attachment of the cells to the microspheres, including the use of extremely high density of cells in suspension. However, this method quickly depleted the media of nutrients and the cell survival was poor. Other techniques included cells that were “dripped” slowly onto the microspheres to increase the physical interaction of the cells with the microsphere or co-culturing the cells and microspheres in a microgravity chamber for several days. While some beta cells would attach to the polymer microspheres, their distribution was uneven, and multiple layers of attached cells were never consistently achieved (FIG. 1B).

### BRIEF SUMMARY OF THE INVENTION

The present invention is directed to an implantable device comprising a substantially planar scaffold comprised of a biomaterial having a major surface, and individual islet cells or small islet cell clusters attached in a multilayer to the surface of the biomaterial scaffold. The individual islet cells or small islet cell clusters are preferably derived from adult intact islets. Cell adhesion molecules (e.g. integrins, cadherins, selecting, and immunoglobulins) may be attached to the scaffold to facilitate attachment of individual islet cells or small islet cell clusters to the scaffold. Further, one or more angiogenesis factors, immunosuppressive agents (including autoimmune suppressors), antibiotics, antioxidants, anti-cytokines, or anti-endotoxins may be controllably released from the scaffold to improve viability of the islet cells and small islet cell clusters.

In another aspect, the biomaterial scaffold is a flexible biomaterial, and may be comprised of a biocompatible and/or biodegradable polymer, such as poly(DL-lactide-co-glycolide) (PLG), polylactic acid (PLA), or poly(lactic-co-glycolic acid) (PLGA).

In still another aspect, the multilayer comprises a combination of insulin-producing beta cells and other islet cell types. The multilayer is preferably about 1-2 to 5 cells thick, and form a multilayer about 10 to 50 microns thick. The multilayer preferably has a substantially uniform thickness such that the cell thickness varies by no more than 1 to 2 cells across the surface of the biomaterial scaffold.

In still another aspect, the individual islet cells or small islet cell clusters are derived from intact adult islets using enzymatic digestion and/or culturing in a calcium-depleted media.

The present invention also provides for a method of forming the implantable device. In particular, techniques for deriving individual islet cells or small islet cell clusters from intact islets are provided (e.g. enzymatic digestion, calcium depletion, or a combination thereof). In addition, methods for attaching the individual islet cells and/or small islet cell clusters are provided, which include centrifuging from a suspension of cells and the use of cell adhesion molecules to improve attachment to the scaffold surface.

In still another aspect, the present invention provides for a method of using the implantable devices of the present invention as a treatment for diabetes. Methods for implanting the devices, and techniques for treatment of diabetes are described.

### BRIEF DESCRIPTION OF THE DRAWINGS

FIGS. 1A and B illustrate previous attempts to grow beta cells on microspherical polymers for implantation into a patient. In the images, an uneven distribution of cells are shown attached to a PLGA microsphere coated with chitosan polymer. A partial monolayer of cells was all that could be obtained after long-term incubation with the beta cells.

FIG. 2 is a graph that compares the cell viability for cultured large rat islets (greater than 125 microns), small islets (less than 125 microns), and dispersed beta cells as a function of time. The decreased viability of large islets is statistically significant (p<0.05) beyond day 3.

FIGS. 3A and B summarize the results of transplantation of small islets (less than 125 microns) or large islets (greater than 125 microns) into diabetic rats. A successful return to euglycemia was observed about 80% of the time when small islets were used, but transplants were unsuccessful in restoring normal plasma glucose levels when the large islets were transplanted. This can be best illustrated by showing the plasma glucose level of the animal in each group 60 days after transplantation. The animals receiving large islets remained hyperglycemic after the transplant, while the rats receiving small islets were euglycemic. * indicates significant difference of 0.01.

FIG. 4 is an islet graft removed from the kidney capsule about eight weeks after transplantation and immunolabeled for insulin. The image on the left panel shows relatively more insulin immunolabeling (red) and an established capillary network in a graft using small islets (less than 125 microns). In contrast, grafts of large islets (greater than 125 microns) showed little insulin immunolabeling and significant fibrosis (right panel). The images are representative from four different animals.

FIG. 5 shows a rat small islet cell cluster stained with dithizone to identify beta cells. Because the confocal aperture was set for an extremely thin Z section, the cells within the subunit, but below the plane of focus, are blurry and do not appear red. However, adjustment in the confocal plane to those cells indicated that they also were clearly stained with dithizone.

FIG. 6 (panel A) shows the live/dead staining of a small islet cell cluster made from an intact adult islet using enzymatic dispersion. This small islet cell cluster is approximately 40 microns in diameter. In the upper right panel of FIG. 6 (panel B), a small islet cell cluster derived by cultivating an intact islet with a calcium depleted media is shown. The small islet cell cluster was unwound or opened so that media was able to surround the cells in the cluster. In FIG. 6 (panel C), a small islet cell cluster derived using both calcium depletion and enzymatic dispersion is shown. The diameter of the fragment was approximately 15 microns. FIG. 6 (panel D) shows individual islet cells derived from a combination of calcium depletion and enzymatic digestion followed by manual pipetting. The red indicates dead cells and green cells are alive. Scale bar in panel B applies to Panels A through C.

FIG. 7 is a schematic representation of the production of a patch having a multilayer of islet cells attached thereto in accordance with the present invention.

FIG. 8 are optical micrographs of beta cell adhesion to (A) chitosan (Mw=100 kDa) and (B) laminin. The inset shows optical and fluorescent micrographs of a beta cell on laminin with cytoch B (green) stain for actin.

FIG. 9 demonstrates the results when layering islet cells onto a polymer patch made of 50:50 PLGA-carboxyl (5.5 kDa). The patches were optically sectioned using a confocal microscope. The images were rendered to obtain the Z section slice shown. The upper panel illustrates a patch with one or two layers of cells, and additional cell layers were then added as shown. Cells were layered onto the scaffold by spinning them in a plate centrifuge at about 3500 rpm for about 10 minutes. The layers remained attached to the polymer scaffold after repeated rinsing.

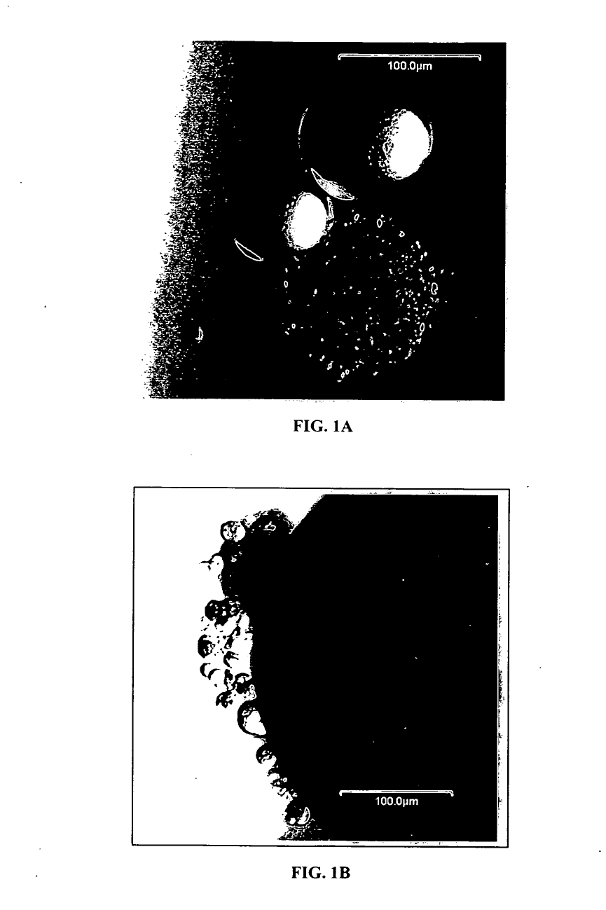

### DETAILED DESCRIPTION OF PREFERRED EMBODIMENT

All patent applications, patents, and publications cited in this specification are hereby incorporated by reference in their entirety. In the case of inconsistencies, the present disclosure, including definitions, will prevail.

As used herein, the term “islet of Langerhans” or “islet” refers to a group of specialized cells in the pancreas that make and secrete hormones. An islet generally contains one or more of the following cell types: (1) alpha cells that make glucagon, which raises the level of glucose (sugar) in the blood; (2) beta cells that make insulin; (3) delta cells that make somatostatin which inhibits the release of numerous other hormones in the body; (4) pancreatic peptide producing PP cells; (5) D1 cells, which secrete vasoactive intestinal peptide; or (6) EC cells which secrete secretin, motilin, and substance P.

As used herein, the term “islet cell” refers to any one of the cells found in an islet. The islet cells used in the present invention are preferably a combination insulin-producing beta cells with other islet cell types.

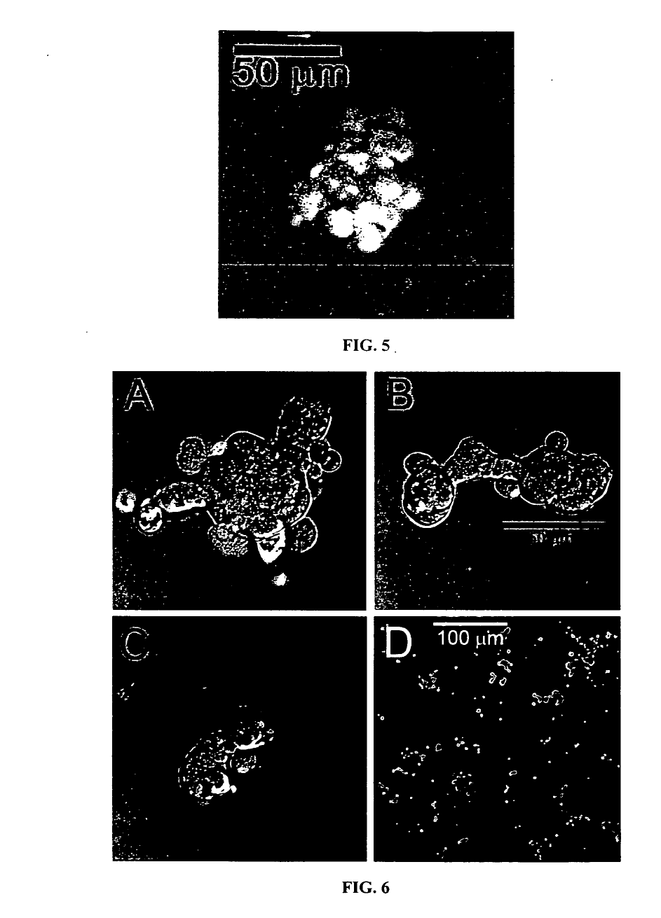

As used herein, the term “small islet cell cluster” refers to a collection of islet cells bounded together, usually less than about 25 cells in the aggregate. The small islet cell cluster preferably has a morphology such that the diffusional barrier for any cell within the cluster (e.g. for nutrients, oxygen, glucose, etc.) is no more than about 7 cells. Typically, the diffusional barrier is less than about 5 cells, and may be as low as 4, 3, or 2 cells. The “small islet cell cluster” preferably comprises beta cells as the predominant cell type, and may optionally include one or more other islet cell types. The small islet cell clusters may have a variety of shapes (e.g., be generally spherical, elongated, or otherwise asymmetrical). Examples of small islet cell clusters are shown in FIGS. 5 and 6(A), 6(B), and 6(C). The “small islet cell clusters” are preferably derived by dispersing intact larger islets isolated from a donor pancreas.

As used herein, materials that are intended to come into contact with biological fluids or tissues (such as by implantation or transplantation into a subject) are termed “biomaterials.” It is desirable that biomaterials induce minimal reactions between the material and the physiological environment. Biomaterials are considered “biocompatible” if, after being placed in the physiological environment, there is minimal inflammatory reaction, no evidence of anaphylactic reaction, and minimal cellular growth on the biomaterial surface. Upon implantation in a host mammal, a biocompatible biomaterial does not elicit a host response sufficient to detrimentally affect the function of the microcapsule; such host responses include formation of fibrotic structures on or around the biomaterial, immunological rejection of the biomaterial, or release of toxic or pyrogenic compounds from the biomaterial into the surrounding host tissue.

The present invention is directed to a method for producing viable individual islet cells or small islet cell clusters for implantation. In one aspect, individual islets cells or small islet cell clusters isolated from non-fetal donor pancreases are attached in a multilayer to the surface of a suitable biomaterial scaffold.

In one aspect, individual islet cells, preferably beta cells, are attached to the biomaterial scaffold. In another aspect, a combination of various islet cell types are attached to the biomaterial scaffold. In still another aspect, small islet cell clusters comprised of two, three, four, five, six, seven, eight, nine, or ten cells are attached to the biomaterial scaffold.

In yet another embodiment, a multilayer of one to two, three, four, or five layers of islet cells are attached to the biomaterial scaffold. The islet cells and small islet cell clusters on the biomaterial scaffold form a multilayer of cells about 10 to 50 microns thick, most preferably about 20 to 40 microns thick.

In one aspect, the multilayer of islet cells preferably has a substantially uniform thickness such that the cell thickness varies by no more than 1-2 cells across the surface of the biomaterial scaffold.

In one aspect, the individual islet cells and/or small islet cell clusters are isolated directly from the pancreas of the donor adult subject and separated from intact islets. Suitable methods for dividing the islets into individual cells and/or small islet cell clusters include enzymatic digestion and metal-based dispersion (calcium depletion), or a combination thereof.

In another aspect, the biomaterial scaffold is comprised of a material that provides for suitable individual islet cell or small islet cell cluster adherence to the scaffold. It is contemplated that various types of materials, including inorganic and organic materials, can be used as the biomaterial scaffold of the present invention. Non-limiting examples of these materials include poly(orthoesters), poly(anhydrides), poly(phosphoesters), poly(phosphazenes), and others. Other non-limiting materials include, for example, polysaccharides, polyesters (such as poly(lactic acid), poly(L-lysine), poly(glycolic acid) and poly(lactic-co-glycolic acid)), poly(lactic acid-co-lysine), poly(lactic acid-graft-lysine), polyanhydrides (such as poly(fatty acid dimer), poly(fumaric acid), poly(sebacic acid), poly(carboxyphenoxy propane), poly(carboxyphenoxy hexane), copolymers of these monomers and the like), poly(anhydride-co-imides), poly(amides), poly(ortho esters), poly(iminocarbonates), poly(urethanes), poly(organophasphazenes), poly(phosphates), poly(ethylene vinyl acetate), and other acyl substituted cellulose acetates and derivatives thereof, poly(caprolactone), poly(carbonates), poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, copolymers, polystyrene, and blends or co-polymers thereof). In certain preferred aspects, the biomaterials include polysaccharides, alginate, hydroxypropyl cellulose (HPC), N-isopropylacrylamide (NIPA), polyethylene glycol, polyvinyl alcohol (PVA), polyethylenimine, chitosan (CS), chitin, dextran sulfate, heparin, chondroitin sulfate, gelatin, etc., and their derivatives, co-polymers, and mixtures thereof. Other suitable biomaterials include those nylon, hyaluronan, polytetrafluoroethylene, polyvinyl formamide, and others described in Vats et al., Scaffolds and biomaterials for tissue engineering: a review of clinical applications, Clin Otolaryngol Allied Sci 28(3): 165-72 (2003); Wang et al., An encapsulation system for the immunoisolation of pancreatic islets, Nat Biotechnol 15(4): 358-62 (1997); Orive et al., Cell encapsulation: promise and progress, Nat Med 9(1): 104-7 (2003), which are incorporated by reference.

In preferred aspects, the biomaterial scaffold is comprised of a biodegradable material. Suitable biodegradable biomaterials include poly(DL-lactide-co-glycolide) (PLG), polylactic acid (PLA), or poly(lactic-co-glycolic acid) (PLGA). PLG is a well-studied polymer for drug delivery and is FDA-approved for a number of in vivo applications. See Berkland et al., Fabrication of PLG microspheres with precisely controlled and monodisperse size distributions, J Control Release May 18, 73(1):59-74 (2001), which is incorporated by reference.

In another aspect, the biomaterial scaffold is coated in whole or in part with a coating that increases the islet and beta cell adhesion. Exemplary coatings include fibronectin, polyethylene glycol acetate, laminin, polyvinyl alcohol (PVA), polyethylene-alt-maleic acid (PEMA), and chitosan (CS).

The scaffold may also have one or more islet cell adhesion molecules (“CAMs”) attached thereto to facilitate individual cell attachment and/or small islet cell cluster attachment to the scaffold. In other applications, CAMs have been previously shown to facilitate cell attachment to polymer for tissue engineering (Dunehoo et al., Cell adhesion molecules for targeted drug delivery, J. Pharm. Sci. 95: 1856-1872 (2006)). Cell adhesion molecules (CAMs) include, but are not limited to integrins (e.g., aᵥb₃, aᵥb₅, LFA-1, VLA-4), cadherins (e.g., E-, P-, and N-cadherins), selectins (e.g., E-, L-, and P-selectins), the immunoglobulin superfamily (e.g., ICAM-1, ICAM-2, VCAM-1, and MadCAM-1), extracellular matrix proteins (e.g., fibronectin, vitronectin, fibrinogen, collagen, laminin, and von Willebrand factor), linear and cyclic cell adhesion peptides and peptidomimetics that are derived from RGD peptides, ICAM-1 peptides, VCAM-1 peptides, cadherin peptides, and LFA-1 peptides. CAMs are essential molecules for tissue regeneration, cell morphology, locomotion, mitosis, cytokinesis, phagocytosis, and the maintenance of cell polarity. CAMs are glycoproteins found on the cell surface that act as receptors for cell-to-cell and cell-to-extracellular matrix (ECM) adhesion. It has been shown previously that cell adhesion molecules such as RGD peptides can help the process of tissue engineering, tissue regeneration, wound healing, reconstructive surgery, neural regeneration, bone grafts, and organ transplantation. In addition, E-cadherin has been shown to be important in β-cell adhesion (Hauge-Evans et al., Pancreatic beta-cell-to-beta-cell interactions are required for integrated responses to nutrient stimuli: enhanced Ca2+ and insulin secretory responses of MIN6 pseudoislets, Diabetes, 48: 1402-1408 (1999)). In one aspect, the cell adhesion molecules are anchored onto the polymer using a covalent bond(s) includes but not limited to a peptide, thioether, disulfide, or ester bond. A spacer molecule may be added between the cell adhesion molecule and the polymer to allow free interactions between the adhesion molecules on the polymer and the cell adhesion receptors on the cell surface. Studies to attached different cells to polymer studded with RGD peptide have shown the optimal spacer between polymer and the RGD peptide is around 11-46 angstroms for the optimal recognition of the RGD peptides by the cell surface receptors. The spacer can be made from but not limited to poly ethylene glycols (PEGs), poly amino acids (e.g., poly-Gly, poly-Lys, poly-Ala), poly amino caproic acids (poly-Aca), and combination of two or three amino acid repeats (e.g., poly-Aca-Gly). In addition to covalent linkage, the cell adhesion molecules can be adsorbed by first attaching the cell adhesion molecule that can be adsorbed into the polymer network of the patch (e.g. electrostatically, hydrophobically, or by other non-covalent interactions) onto the polymers prior to attaching the islet cells.

In another aspect, the biomaterial scaffold has a shape that facilitates attachment of the individual islet cells or small islet cell clusters to its surface. The scaffold typically has a substantially planar surface, such as that on a patch or disk. In the preferred embodiment, the biomaterial scaffold comprises a substantially planar flexible patch material.

The biomaterial scaffold has a size suitable for attachment of individual islet cells or small islet cell clusters. For example, in one aspect, the planar patch typically has dimensions on the order of about 0.2 to 3 centimeters. The thickness of the patch is typically on the order of about 50 microns to 1 centimeter.

In yet another aspect, the biomaterial scaffold can controllably release one or more growth factors, immunosuppressant agents, antibiotics, antioxidants, anti-cytokines, anti-endotoxins, T-cell adhesion blockers, angiogenesis factors, nutrients, or combinations thereof.

Exemplary growth factors include, epiregulin, epidermal growth factor (“EGF”), endothelial cell growth factor (“ECGF”), fibroblast growth factor (“FGF:), nerve growth factor (“NGF”), leukemia inhibitory factor (“LIF”), and bone morphogenetic protein-4 (“BMP-4”), hepatocyte growth factor (“HGF”), vascular endothelial growth factor-A (“VEGF-A”), cholecystokinin octapeptide, insulin-like growth factor, and even insulin itself. See generally Miao et al., In vitro and in vivo improvement of islet survival following treatment with nerve growth factor, Transplantation February 27; 81(4):519-24 (2006); Ta et al., The defined combination of growth factors controls generation of long-term replicating islet progenitor-like cells from cultures of adult mouse pancreas, Stem Cells, Mar. 23, 2006; Johannson, Islet endothelial cells and pancreatic beta-cell proliferation: studies in vitro and during pregnancy in adult rats, Endocrinology May; 147(5):2315-24 (2006), Epub Jan. 26, 2006; Kuntz et al., Effect of epiregulin on pancreatic beta cell growth and insulin secretion, Growth Factors. December 23(4):285-93 (2005); Vasadava, Growth factors and beta cell replication, Int J Biochem Cell Biol. 38(5-6):931-50 (2006), Epub August 31 Review (2005); Kuntz et al., Cholecystokinin octapeptide: a potential growth factor for pancreatic beta cells in diabetic rats, JOP. November 10; 5(6):464-75 (2004).

Exemplary immunosuppressant agents are well known and may be steroidal or non-steroidal. Preferred steroidal agents are prednisone. Preferred non-steroidal agents include those in the so-called Edmonton Protocol: sirolimus (Rapamune, Wyeth-Ayerst Canada), tacrolimus (Prograf, Fujisawa Canada), and anti_IL2R daclizumab (Zenapax, Roche Canada). Other immunosuppressant agents include 15-deoxyspergualin, cyclosporine, rapamycin, Rapamune (sirolimus/rapamycin), FK506, or Lisofylline (LSF).

Exemplary antibiotics useful for the practice of this invention include but are not limited to amoxicillin, penicillin, sulfa drugs, erythromycin, streptomycin, tetracycline, chlarithromycin, ciproflozacin, terconazole, azithromycin, and the like.

Various antioxidants are known to those skilled in the art. Particularly preferred are molecules including thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hydroxyanisole (BHA), vitamin K, and the like. Glutathione, for example, may be used in a concentration range of from about 2 to about 10 mM. See, e.g., U.S. Pat. Nos. 5,710,172; 5,696,109; and 5,670,545.

Suitable anti-cytokines well known in the art and include dimethylthiourea (about 10 mM), citiolone (about 5 mM), pravastatin sodium (PRAVACHOL, about 20 mg/kg), L-N^{G}-monomethylarginine (L-NMMA, 2 mM), lactoferrin (about 100 μg/ml), 4-methylprednisolone (about 20 μg/ml), and the like.

Anti-endotoxins are also known in the art and include L-N^{G}-monomethylarginine (L-NMMA, about 2 mM), lactoferrin (about 100 μg/ml), N-acetylcysteine (NAC, about 1 mM), adenosine receptor antagonists such as bamiphylline (theophylline), and anti-lipopolysaccharide compounds such as echinomycine (about 10 nM), and the like.

In still another aspect, a T-cell adhesion blocker is provided to the implanted biopolymers containing islet cells to suppress immune reaction. Addition of these blockers prevents rejection of islet transplantation. T-cell adhesion blockers have been shown suppress T-cell activation and immune response in organ transplantation and autoimmune diseases (see Yusuf-Makagiansar et al., Inhibition of LFA-1/ICAM-1 and VLA-4/VCAM-1 as a therapeutic approach to inflammation and autoimmune diseases, Medicinal Chemistry Reviews 22, 146-167 (2002); Anderson and Siahaan, Targeting ICAM-1/LFA-1 interaction for controlling autoimmune diseases: Designing peptide and small molecule inhibitors, Peptides 24, 487-501 (2003)). The T-cell adhesion blockers include but are not limited to (a) monoclonal antibodies to ICAM-1, LFA-1, B7, CD28, CD2, and VLA-4, (b) soluble protein and its fragments such as ICAM-1, VCAM-1, MadCAM-1, (c) RGD peptides and peptidomimetics, (d) VCAM-1 peptides and peptidomimetics, (e) ICAM-1 peptides and peptidomimetics, and (f) LFA-1 peptides and peptidomimetics. In addition, peptides (e.g. GAD₂₀₈₋₂₁₇) derived from glutamic acid decarboxylase 65 (GAD65) and the GAD bifunctional peptide inhibitor (GAD-BPI) have been shown to induce immunotolerance and suppress islet infiltration by T-cells (insulitis). GAD₂₀₈₋₂₁₇ has been show to block the activation of T-cells that attack the beta cells in non-obese diabetes (NOD) mice by modulating the TCR-MHC-Ag complex formation (Signal-1) during T-cell:APC interaction (Tisch et al., Induction of GAD65-specific regulatory T-cells inhibits ongoing autoimmune diabetes in nonobese diabetic mice, Diabetes 47: 894-899 (1998)). The preferred GAD-BPI comprises GAD₂₀₈₋₂₁₇ linked to a portion of the LFA-1 peptide (sequence EIAPVFVLLE-[Ac-G-Ac-G-Ac]-ITDGEATDSG), and has been shown to block T-cell activation and insulitis in NOD mice as set forth in Murray et al., Published U.S. Patent No. 2005/0107585 entitled “Signal-1/signal-2 bifunctional peptide inhibitors,” which is incorporated by reference. Thus, these molecules may be co-administered to prevent rejection of the islet transplant. These molecules may also be delivered via controlled release mechanisms to prevent rejection of the islet transplant. Thus, the molecules may be trapped inside the biomaterial scaffold before the beta cells are attached to the scaffold.

The controlled release of such agents may be performed by using the protocols set forth in Raman et al., Modeling small-molecule release from PLG microspheres: effects of polymer degradation and nonuniform drug distribution, J. Control Release. March 2; 103(1):149-58 (2005); Berkland et al., Precise control of PLG microsphere size provides enhanced control of drug release rate, J. Control Release. July 18; 82(1):137-47 (2002); Schwendeman, Recent advances in the stabilization of proteins encapsulated in injectable PLGA delivery systems, Crit. Rev. Ther. Drug Carrier Syst., 19(1):73-98 (2002); Sershen et al., Implantable, polymeric systems for modulated drug delivery, Adv. Drug Deliv Rev 5; 54(9):1225-1235 (2002), all of which are incorporated by reference.

Additional aspects of the invention, together with the advantages and novel features appurtenant thereto, will be set forth in part in the description and examples which follow, and in part will become apparent to those skilled in the art upon examination of the following, or may be learned from the practice of the invention. The objects and advantages of the invention may be realized and attained by means of the instrumentalities and combinations particularly pointed out in the appended claims.

### EXAMPLE 1

### Size of Islet Impacts Viability and Transplantation Success

This example investigated how islet size affected transplantation success in rats. In this example, techniques for isolating islets are described, and cell viability was measured. Both large islets (greater than 125 microns) and small islets (less than 125 microns) were transplanted in order to assess the effect of islet size on transplantation success. As discussed below, small rat islets are superior to large islets in in vitro function and in in vivo transplantation outcomes. These experiments are also described in MacGregor et al., Small rat islets are superior to large islets in in vitro function and in transplantation outcomes, Am J Physiol Endocrinol Metab. May; 290(5):E771-9 (2006), which is incorporated by reference in its entirety.

Rat Islet Isolation.

To isolate large and small islets, adult male DA rats were anesthetized by intraperitoneal injection of a mixture of ketamine and xylazine. The peritoneal cavity was exposed and the pancreatic ductal connection to the intestine clamped. The pancreas was cannulated in situ via the common bile duct, and distended by pumping a cold solution of collagenase into the duct. Collagenase (CLS-1, Worthington Biochemical Corp, Lakewood, N.J.) was dissolved in 20 ml of Leibovitz L15 at 450 U/ml. Subsequently the distended pancreas was excised, transferred to 50 ml centrifuge tubes, and incubated for about 20-30 minutes with gentle tumbling in a 37° C. incubator. Following incubation, the tube was gently shaken to dislodge islets. The contents of the tube were placed in diluted ice-cold Hank's Balanced Salt Solution (“HBSS”) containing 10% of newborn calf serum. The digest was allowed to settle at 1×g and the supernatant removed. More HBSS/serum was added and the process repeated. The washed digest was passed through a 500 micron stainless steel screen and sedimented about 1 minute at 300×g in a refrigerated centrifuge. The pellet was mixed with 10 mL of 1.110 gm/mL Histopaque (density=1.1085, Sigma Diagnostics Inc., St. Louis, Mo.) and centrifuged 10 minutes at 800×g. The islets floating on the gradient were collected and sedimented separately, then placed into Ham's F12 culture medium containing 10% of fetal bovine serum and put into a 37° C. culture chamber containing 5% CO₂.

Yield

For yield measurements, triplicate samples of each batch of islets were examined, each comprising approximately 2% of the islet fraction. Individual islets were counted and their diameters measured. For irregular-shaped islets, 3 to 4 diameter measurements were taken at different locations on the islet and the average used. Islet volumes were calculated and converted to islet equivalents for the sample and the entire islet fraction. Small islets were defined as those having a diameter of less than about 125 microns compared to large islet with a diameter of about 125 microns or greater.

To separate small islets from large islets, fresh islets or islets cultured overnight were sedimented and then placed in 1-2 ml of L15 medium. The islets were then quickly layered over a single-step gradient of 5% BSA in L15. Sedimentation at 1×g was permitted to occur for an empirically set period of time until large islets were observed in the bottom of the tube. At that point the top two milliliters (without BSA) of the gradient was discarded, and all but the bottom 2 ml was carefully removed to define the small islet population. The sedimented islets and those in the bottom 2 milliliters were combined as the large islet fraction. Gradients were repeated if needed to optimize the separation of large and small islets. Final islet fractions were sedimented and place into culture in a 1:1 mixture of Ham's F12 and glucose-free RPMI 1640 (glucose=5 mM) until glucose sensitivity experiments were performed.

Viability

To test viability, islets were placed in a 500 μl volume of L-15 media with live/dead fluorophores, Sytox (Molecular Probes, 1 μM) and Calcein (Molecular Probes, 0.5 μM), and incubated for about 15 to 30 minutes at 37° C. Islets were rinsed with phosphate buffered saline (PBS) consisting of (in mM): 137 NaCl, 2.7 KCl, 4.3 Na₂HPO₄ and 1.4 KH₂PO₄, pH 7.4 and placed in the Attofluor Chamber (Molecular Probes) on the Olympus Fluoview 300 confocal microscope housed in the Diabetes Research Laboratory. Images were acquired using 40× or 60× objectives. All images were collected within 20 minutes of removal of the islets from the media. Three simultaneous images were collected for each islet using He:Ne and Argon lasers and a third bright-field image.

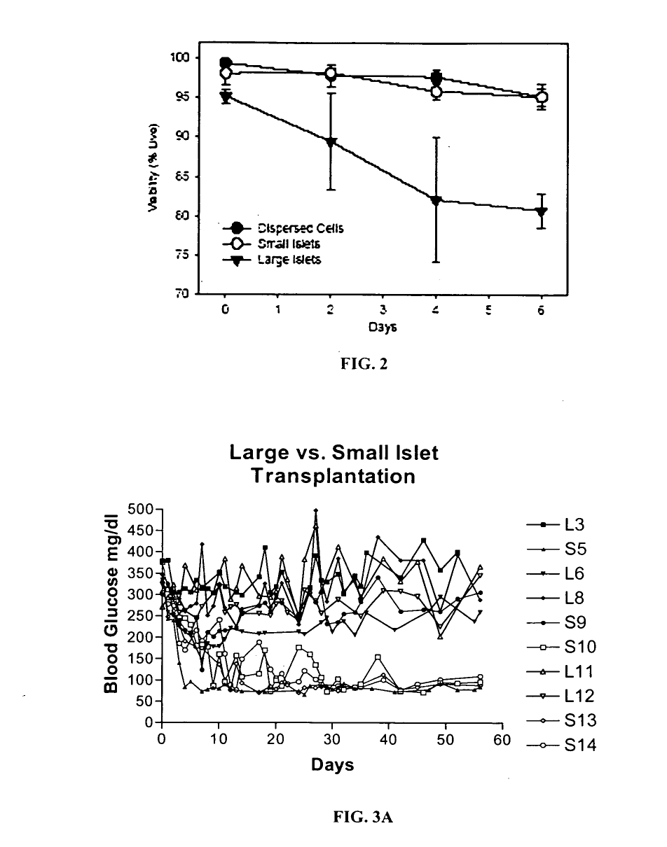

As shown in FIG. 2, large intact islets (greater than 125 microns), whether human or rat, maintained in culture typically exhibit a significant percentage of necrotic (12.6%) and apoptotic (6.3%) cells after only four days with cell death increasing over time. Smaller islets (less than 125 microns) exhibited extended viability, but still showed precipitous cell death at later time points (beyond one week). The viability of these small islets was followed for up to one week, and it was found that they maintain high viability percentages from 99 to 86%. This is in comparison to intact large islets, which have viability levels that fall to below 50% after several days in culture. As shown, in FIG. 2, individually dispersed islet cells maintain a high viability profile in culture similar to the small intact islets.

Live/dead analysis was completed by identifying the islets in the field and encircling the regions of interest. Background fluorescence was subtracted from all images. Viability percentages were calculated by developing hue histograms using Photoshop (Adobe) from the fields of interest and calculating the total pixels in the green hue (live) and red (dead). The ratio representing the live cells divided by the total islet area was calculated as the percent live value. Islet diameters and perimeters were calculated using Scion software so that viability values could be categorized according to the size of the islet.

Transplant Studies

The effect of islet size on transplantation success was also investigated. In the experiments, diabetes was induced in the recipient animals by injecting streptozotocin (65 mg/kg) intraperitoneally (1 injection). When blood glucose levels are greater than 250 mg/dl for three consecutive days, the rats were considered diabetic.

Rats were anesthetized with pentobarbital 45 mg/kg. After the rat was shaved and cleaned with betadine scrub, an incision was be made in the body wall on the left flank. The kidney was delivered into the wound, and a small incision was made in the kidney capsule. The large or small islets were placed under the capsule using a small bore pipette. The kidney was placed back into original position and the incision closed with wound clips. Beef/porcine zinc-insulin (NPH Iletin I) injections (2 times/day) were given to recipients for three days post-islet transplant to reduce the stress of hyperglycemia on the newly transplanted islets.

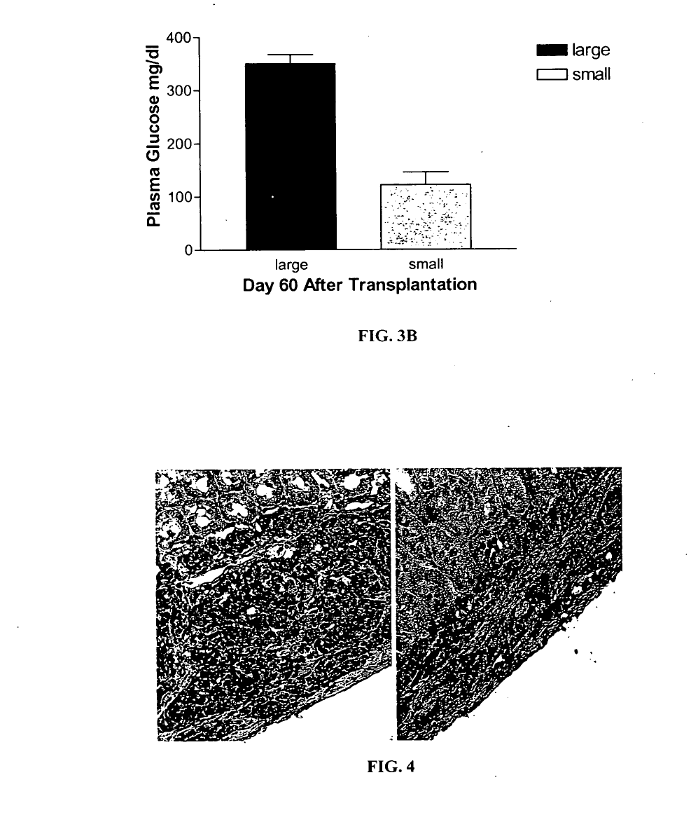

Transplantations of the large or small rat islets were completed (n=10 transplantations/group). The streptozotocin-induced diabetic DA rats received a marginal mass (1000IE) of either large (greater than 150 microns) or small (less 125 microns) syngeneic islets under the kidney capsule. Blood glucose levels were monitored for eight weeks. FIGS. 3(A) and 3(B) show the results from the first five transplants for each group. All of the recipients of large islets remained hyperglycemic after transplantation (10 of 10). In contrast, 8 of 10 recipients of small islets had blood glucose levels close to or at normal levels 7-10 days after transplantation, which remained normal for the entire eight-week period.

Islet grafts from the kidney capsule were removed eight weeks after transplantation. The tissue was fixed and immunolabeled for insulin. FIG. 4 (left panel) shows the graft from an animal that received small islet transplantation and was euglycemic for the eight weeks. There was substantial staining for insulin in the graft. In contrast, FIG. 4 (right panel) the animal that received the transplantation of large islets continued to be hyperglycemic for the eight week period and showed little immunolabeling for insulin in the grafts.

Together, the foregoing experiments show that smaller islets (less than 125 microns) were superior to large islets (more than 125 microns) in viability, in vivo functional assays, and in transplant outcomes. In addition, an average pancreas yielded about three times more small islets than large islets, and the smaller islets were approximately 20% more viable. Most importantly, the small islets were far superior to large islets when transplanted into diabetic animals.

### EXAMPLE 2

### Conversion of Large Islets into Individual Islet Cells or Small Islet Cell Clusters

This example illustrates methods for fragmenting or dispersing intact islets into a small islet cell clusters (such as the cluster shown in FIG. 5) and individual islet cells. The small islet cell cluster in FIG. 6(A) was created using a conventional enzymatic digestion, while the small islet cell cluster in FIG. 6(B) was formed with graded calcium depletion. As the image in FIG. 6(A) illustrates, enzymatic dispersion breaks the islet down into small islet cell clusters, but it does not “open” the cluster up so the cells on the interior of the cluster have a diffusional barrier that is several cells thick. In contrast, for small islet cell clusters formed using calcium depletion (FIG. 6(B)), the cluster has an “open” morphology such that there is a smaller diffusional barrier for each cell of the when the small islet cell cluster. It is anticipated that a combination of enzymatic digestion and calcium depletion may also be used to covert intact islets into small islet cell clusters, which is shown in FIG. 6(C).

a. Enzyme Digestion

Different enzyme cocktails can be used to fragment intact islets into small islet cell clusters and individual islet cells. Exemplary enzymatic digestion methods are disclosed in U.S. Pat. No. 6,783,954, which is incorporated by reference. In this example, twelve enzyme cocktails were used with varying degrees of success, including one cocktail which included papain.

To isolate pancreatic islets, Sprague-Dawley rats were anesthetized by an intraperitoneal injection of ketamine and xylazine. The peritoneal cavity was exposed and the pancreatic ductal connection to the intestine clamped. The pancreas was cannulated in situ via the common bile duct, and distended by pumping a cold solution of collagenase into the duct. Subsequently, the distended pancreas was excised, transferred to centrifuge tubes, and incubated for about 30 minutes with gentle tumbling in a 37° C. The washed digest was passed through a screen and sedimented in a refrigerated centrifuge. The pellet was mixed with Histopaque (density=1.1085, Sigma Diagnostics Inc., St. Louis, Mo.) and centrifuged. The islets were then placed into Ham's F12 culture medium containing 10% of fetal bovine serum and put into a 37° C. culture chamber containing 5% CO₂.

The standard protocol for beta cell isolation included incubating intact islets (isolation using techniques described herein) in Hanks Balanced Salt Solution (“HBSS”) with 4.8 mM Hepes. See Balamurugan et al., Flexible management of enzymatic digestion improves human islet isolation outcome from sub-optimal donor pancreata, Am J Transplant 3(9): 1135-42 (2003). For enzymatic digestion, a final 9 ml of Hank's balanced salt solution containing 1 ml of papain (50 units/ml) was added to the islets. Islets were initially pipetted up and down gently to ensure complete rinsing. Islets were allowed to settle to the bottom of the tube and most of the supernatant was removed. Islets in the enzyme were rotated slowly (about 10 prm) for about 30 minutes at 37° C. At this point, small islet clusters were formed with some single dispersed cells, and removed from the solution. Typically, the cells were transferred to CMRL 1066 or Memphis SMF as the final culture media.

Cells were stained with dithizone to identify the beta cells within the clusters as generally shown in FIGS. 5 and 6(A) (enzyme).

b. Metal-Based Fragmentation

Intact islets may also be fragmented into small islet cell clusters and individual islet cells using a metal-based fragmentation approach. The interesting finding of metal-based fragmentation is that the resulting small islet cell clusters are less-compact or have an “open” morphology. Cell adhesion molecules, such as E-cadherin, hold the islet together, but require divalent metals to function. See Hauge-Evans et al., Pancreatic beta-cell-to-beta-cell interactions are required for integrated responses to nutrient stimuli: enhanced Ca2+ and insulin secretory responses of MIN6 pseudoislets, Diabetes 48(7): 1402-8 (1999). Thus, culturing islets in calcium-free media for about one hour results in a “loosening” and fracturing of the islet structure (see FIG. 6(B)) in comparison to utilizing enzymes alone, which yields a denser islet structure (see FIG. 6(A)). Further, after “loosening” the islets using calcium depletion, the remaining clumps of beta cells are more easily dispersed by traditional enzymes (see FIG. 6(C)).

The details of the metal-based fragmentation are as follows. To obtain individual islet cells and small islet cell clusters, the islets were in calcium-magnesium free Hanks Balanced Salt Solution+4.8 mM Hepes. After incubation at about 37° C. for about 30 minutes, the cells were pipetted, dispersing them into small islet cell clusters or single cells. The cells were transferred to CMRL 1066 as the final culture media. When necessary, the small islet cell clusters or beta cells were identified with dithizone. See Mythili et al., Culture prior to transplantation preserves the ultrastructural integrity of monkey pancreatic islets, J. Electron Microsc (Tokyo) 52(4): 399-405 (2003).

As shown in FIG. 6(B), the small islet cell clusters derived by calcium depletion alone had an irregular tubular arrangement, which may be optimal for perfusion of the core of the cluster. In addition, the clusters derived from metal-based dispersion take only about one hour to produce, while the enzyme approach to fragmentation can take up to 48 hours.

c. Combination of Enzymatic Digestion and Metal Dispersion

Experiments were also performed using a combination of enzymatic digestion and metal depletion as a dispersion technique. Intact islets were rinsed with 9 ml of Hank's balanced salt solution (without calcium or magnesium) with 4.8 mM Hepes. Islets were initially pipetted up and down gently to ensure complete rinsing. Islets were allowed to settle to the bottom of the tube and most of the supernatant was removed. The islets could be repeatedly washed to remove all calcium and magnesium.

A final 9 ml of calcium and magnesium-free Hank's balanced salt solution containing 1 ml of papain (50 units/ml) was added to the islets. Islets in the enzyme were rotated slowly (10 prm) for 30 minutes. At this point small islet clusters could be removed from the solution. Strong pipetting 2-3 times at a moderate speed resulted in single cells.

Cells were centrifuged for 5 minutes at 1500 rcf, 25° C. Single cells were resuspended using the appropriate culture media (depending on the subsequent assays). Cells were stored in an incubator at 37° C. and 5% CO₂. As shown in FIG. 6(C), combination of the enzyme and calcium depletion method results in a small islet cell clusters. Moreover, the combination was an overall faster dispersion protocol, but caution must be used to avoid over-digested and damaged cells.

In these experiments, YO-PRO-1 and propidium iodide (Vibrant Apoptotic Assays, Molecular Probes) were used to determine necrotic and apoptotic cells. For the assay, cells were placed with PBS in the Attofluor Chamber (Molecular Probes) on the Olympus Fluoview 300 laser confocal microscope. All images were collected within 20 minutes of removal of the cells from the media. Three simultaneous images were collected for each islet using He:Ne and Argon lasers and a third bright-field image. Live/dead analysis was completed by identifying the cells in the field using transmitted light. Green cells indicate apoptosis, while yellow/red indicates necrotic cell death. Cells lacking fluorescence emission were live. The fluorescence images were overlaid with the transmitted-light image (gray).

### EXAMPLE 3

### Preparation of Individual Islet Cells and Small Islet Clusters onto a Patch Biomaterial Scaffold

The foregoing examples indicate that small islet cell clusters and even individual beta cells should represent the highest achievable free surface area for transporting oxygen, glucose, etc. Thus, in this example, individual islet cells or small islet cell clusters were templated onto a biomaterial scaffold material, such as a patch as generally shown in FIG. 7, to form a multilayer of islet cells.

Screening of Scaffold Materials

In this example, optimization of various biomaterials useful for preparing the scaffolds of the present invention were investigated by measuring the relative adhesion of the islet cells to the biomaterial. It is preferable that the scaffold material be easy to handle without dissociating the tissue and biomaterial backing to enable facile implantation. Table 1 illustrates a wide variety of biomaterials which were selected for interactions with beta cells. Several of these materials possess a history of use as implants.

In a typical experiment, 1% stock solutions of the listed biomaterials first were prepared. Most materials dissolved in deionized water at neutral pH. Chitosan required a lower pH of about 5.5 to dissolve (hydrochloric acid was used) and other materials required organic solvents; for example Cellform™ in ethanol and poly(DL-lactic-co-glycolic) acid (PLGA) in dichloromethane. Polymers normally soluble in water (e.g. dextran sulfate, alginate, etc.) can be cross-linked to form the film matrix. Approximately 25 μL of each stock solution was added to three individual wells in 96-well plates and left to evaporate or vacuum dried, thus, depositing a thin biomaterial film at the bottom of each well. Residual solvent content is miniscule and did not induce toxicity in cells. Several proteins offered commercially to promote cell adhesion on well plates (e.g. fibronectin, laminin, etc.) were prescreened for cell adhesion as well.

A dilute suspension of beta cells was incubated in the 96-well plates overnight and washed three times to remove unbound beta cells. The beta cell suspension was homogeneous and equal aliquots per well were assumed to contain a similar quantity of beta cells. All cell counts were normalized to cell counts from wells that did not include a biomaterial film. In general, mildly hydrophobic polymers performed well for adhering beta cells (Table 1).

TABLE 1
Relative beta cell adhesion of selected biomaterials

| Biomaterial | | Relative cell adhesion |
|---|---|---|
| Empty well (control) | | 1 |
| 50:50 PLGA-carboxyl | Mw = 5.5 kDa | 9.8 ± 0.9 |
| Laminin | | 8.7 ± 0.6 |
| Dextran Sulfate | Mw = 500 kDa | 7.4 ± 3.0 |
| 50:50 PLGA-methylester | iv = 0.31 dL/g | 6.8 ± 0.7 |
| Polyvinypyrrolidone | | 5.8 ± 1.2 |
| Dextran Sulfate | Mw = 8 kDa | 5.4 ± 1.0 |
| 50:50 PLGA-methylester | iv = 0.9 dL/g | 5.2 ± 0.8 |
| 50:50 PLGA-methylester | iv = 0.58 dL/g | 4.4 ± 0.7 |
| Pluronic | | 4.0 ± 1.5 |
| 50:50 PLGA-carboxyl | iv = 0.12 dL/g | 3.9 ± 0.7 |
| Polyethylenimine | Mw = 25 kDa | 3.8 ± 0.2 |
| Fibronectin | | 3.7 ± 0.7 |
| PEG acrylate | | 3.1 ± 0.5 |
| Chitosan | Mw = 15 kDa | 3.1 ± 0.1 |
| Collagen IV | | 2.9 ± 1.4 |
| PEG | Mw = 8 kDa | 2.8 ± 1.1 |
| Alginate | | 2.4 ± 1.2 |
| Gelatin | | 2.0 ± 0.2 |
| Heparin | | 1.7 ± 0.2 |
| Cellform ™ | | 1.7 ± 0.7 |
| Chitosan | Mw = 100 kDa | 1.5 ± 0.7 |
| Polyethylenimine | Mw = 800 Da | 1.2 ± 1.0 |
| Polyvinypyrrolidone | | n.d. |
| Poly(vinyl alcohol) | | n.d. |
| Poly(acrylic acid) | | n.d. |

iv = inherent viscosity

Cell adhesion was determined by counting the number of attached cells 24 hours after plating on the biomaterial and following three washes. The counts were normalized to the number of cells that attach to a well bottom lacking a biomaterial (see empty well, control) using the following calculation: number of cells attached in the well of interest/number of cells in empty well. Each experiment was repeated in triplicate.

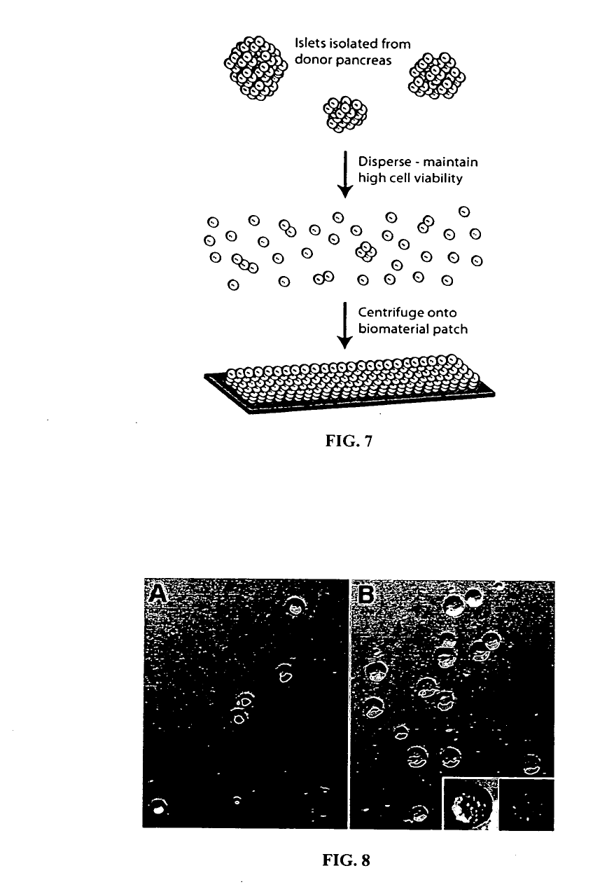

In general, mildly hydrophobic polymers performed well for adhering beta cells. Optical micrographs indicated that cell morphology was also affected by the biomaterial. Beta cells on chitosan (MW=100 kDa) exhibited a smooth, rounded surface while beta cells on laminin demonstrated a spread and ruffled morphology (see FIG. 8). Fluorescent staining of actin in beta cells on the laminin substrate revealed strongly fluorescent cytoskeleton focal points suggesting firm cell adhesion.

Preparation of Islet Cell Patch

In this example, the islet cells were bound to a biomaterial scaffold patch comprising PLGA. In vascularized islets of Langerhans, the average beta cell is no more than about 25 microns away from a blood vessel. See Wayland, Microcirculation in pancreatic function, Microsc Res Tech 37(5-6): 418-33 (1997). Because beta cells are about 10 microns in diameter, it is anticipated that cell layer thickness of about three cells would most accurately mimic the native beta cell environment.

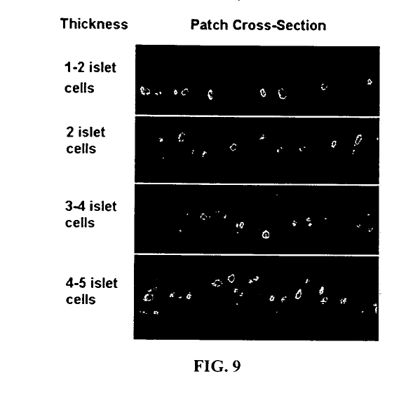

In general, islets were isolated from a rat pancreas and dispersed into single cells or small cell clusters as described previously. Islet cells and small islet cell clusters in HBSS media (0.5 ml) were added to each well and allowed to culture onto the biomaterial for 3 to 4 hours. Plates with biopolymers in the wells were spun in a centrifuge at room temperature at about 3500 rpm for about 10 minutes to assist the cells in attaching to the biopolymer. Half of the media was removed from each well, replaced with media containing a fresh islet cell or small islet cell cluster suspension, and allowed to attach (either by gravity or by centrifugation). This was repeated three times. Results of these experiments are shown FIG. 9. Additional layers of islet cells can be attached to the patch of polymer following repeated washing when the centrifugation method was employed, compared to cells cultured on polymers without centrifugation. About three to five layers of cells remain consistently attached to 50:50 PLGA at 0.58 dL/g (in HFIP) or 0.9 dL/g polymer with repeated media changes. To control the thickness of the beta cell layer, either the volume of cell culture added to each well and/or the number of aliquots added to each well in repeated deposition cycle can be controlled.

### EXAMPLE 4

### Prophetic Testing of Islet Cells on Biomaterial Scaffold

In this example, biomaterial patches having a multilayer of islet cells attached thereto will be further investigated. Viability measures and insulin production assays will be performed. In addition the device will be investigated as an implantable device for the treatment of diabetes.

Viability measurements. Apoptosis versus necrosis experiments will be completed as previously. The percentage live cells will be calculated per cross sectional area of the beta cell layers for comparison to native islets on days 0, 1, 3, 7, 14, and 30 for three samples. Data will be plotted as percent viable cells versus time and we will determine if a statistically significant difference exists between the viability trends for different numbers of beta cell layers using a t-test. In addition, recording of the percentage of cell death attributed to necrosis or apoptosis will be made.

Insulin production assays. Insulin production will be measured using static incubation (ELISA) under conditions of low glucose (3 mM), high glucose (30 mM), and high glucose/depolarization (25 mM K+) (Dean 1989). Each well in 12-well plates will be preincubated with fresh media at 37° C. and 5% CO₂. For experimental measurement, the various beta cell patches will be incubated for 2 hours in fresh media containing either 3 or 30 mM glucose. One additional group of wells is incubated in 30 mM glucose, containing 25 mM KCl with appropriately reduced NaCl. Each patch type will be evaluated in triplicate for each condition tested. Media samples will be assayed for insulin content using an ELISA immunoassay. The results will be expressed as averages of the triplicate samples with standard deviation and compared using a t-test for statistical significance. MacGregor et al., Small rat islets are superior to large islets in in vitro function and in transplantation outcomes, Am J Physiol Endocrinol Metab. 290(5): E771-779 (2006).

Implantation of patches and islets. Diabetes will be induced in adult recipient Diabetes Resistant BioBreeding (DRBB) Worcester rat is a model of autoimmune diabetes that parallels type 1 diabetes in humans. Four-week old rats will be purchased from Biomedical Research Models, Inc. Animals will be randomly divided into 2 groups: patch recipients and islet recipients (6 per group). For the induction of diabetes the DRBB rats will be treated with a combination of anti-RT6 monoclonal antibody (DS4.23 hybridoma (kindly provided by Dr. Dale L. Greiner, University of Massachusetts Medical Center; 2 ml tissue culture medium injected 5 times/week) and non-specific immune system activator poly I:C (Sigma; 5 ug/g of body weight injected 3 times/week). The injections will be given over a 3-week period. On the date of repeated hyperglycemia (blood glucose levels>250 mg/dl for 3 consecutive days), the animals will be considered diabetic and the treatment discontinued (Searls 2004). With this method, 95% of the rats become diabetic by the end of the 3rd week. Implantation of beta cell patches and islets will be done in the kidney subcapsule. DA (Dark Aqouti) rats will serve as beta cell donors. Rats will be anesthetized with pentobarbital (45 mg/kg) and the kidney delivered to an incision made in the body wall on the left flank. A moderate incision will be made in the kidney capsule, and the beta cell patch placed under the capsule. A minimum of 4 patches with variable biomaterial and/or cell layer thickness will be implanted. Islet implants typically require a smaller incision and infusion through a small bore pipette. Recipient groups will receive either 1000 or 2000 IE of islets for transplants or an equivalency of beta cells on the patch substrate. Significant improvement in performance (patch type versus islets) should be detectable if the minimum necessary islets for success (1000 IE) are transplanted and compared to a higher islet volume (2000 IE). Beef/porcine zinc-insulin (NPH Iletin I) injections (2 times/day) will be given for 3 days post-islet transplant to reduce the stress of hyperglycemia.

In vivo determination of glycemia. The blood glucose of rats will be monitored for 4 weeks to determine whether the patch or islet implants can induce euglycemia. The glycemic control of the animals will be followed by taking blood glucose measurements daily. Plasma glucose levels will be monitored by obtaining blood samples from the tail on a daily basis for the first 3 weeks, and then 2 times/week using the Freestyle glucose meter (TheraSense). Generally reversal of diabetes is achieved within 24 hours of islet transplantation, similar outcomes should be achieved with the patches.

Analysis of explanted beta cell patches. The patches or islets will be retrieved after 14 or 30 days for immunostaining (insulin and glucagon), viability measurement, and detection of apoptosis. In some cases, rats achieving euglycemia will be maintained for 8 weeks before analysis. Immunohistochemistry on the sections will be completed using antibodies for insulin and glucagon. Images will be processed using colorimetric analysis to determine the cross-sectional area positive for each of the stains. Negative control slides will be prepared and analyzed. Initially, we will use a dithizone stain to identify beta cells. DNA-fragmentation, indicative of cellular apoptosis, will be completed using terminal deoxynucleotidyl transferase (TdT)-mediated dUTP nick-end labeling (TUNEL) assay. Patches or islets will be prepared for histology using 10% formalin embedded in paraffin as we have previously done. The TUNEL kit (In Situ Cell Death Detection Kit, Roche Diagnostics) will be used to label the histological sections. The patches and islets will be analyzed both for the number and distribution of TUNEL+ cells by a blinded researcher. Images of histological sections will be reconstructed into full 3D images of islets. In this way, apoptotic cells throughout single islets can be identified. Sections will be counterstained with hematoxylin and visualized under the light microscope. To identify the insulin-secreting cells within the islets, anti-insulin antibody will be used to label samples and detected with a rhodamine secondary antibody. We anticipate collecting a minimum of 10 islets/rat post transplantation for apoptosis analysis. Negative control slides will be prepared as necessary. In addition to TUNEL analysis, patches will be fixed for subsequent electron microscopy using the core microscopy facility. Identification of beta cell layers and of infiltrating cells will be conducted in this manner.

### REFERENCES

The following references, to the extent that they provide exemplary procedural or other details supplementary to those set forth herein, are specifically incorporated herein by reference.
Balmurgan A N, Chang Y, Fung J J, Trucco M, and Bottino R., Flexible management of enzymatic digestion improves human islet isolation outcome from sub-optimal donor pancreata, Am J Transplantation 3: 1135-1142, (2003).
Chan C B, Saleh M C, Purje A, and MacPhail R M, Glucose-inducible hypertrophy and suppression of anion efflux in rat beta cells, J Endocrinol 173: 45-52, (2002).
Chan C B and Surette J J, Glucose refractoriness of beta-cells from fed fa/fa rats is ameliorated by nonesterified fatty acids, Can J Physiol Pharmacol 77: 934-942, (1999).
Cui Y-F, Ma M, Wang G-Y, D-E. H, Vollmar B, and Menger M D, Prevention of core cell damage in isolated islets of Langerhans by low temperature preconditioning, World J Gastroenter 11: 545-550, (2005).
Dean et al., Comparison of insulin autoantibodies in diabetes-related and healthy populations by precise displacement ELISA, Diabetes (1989).
Dufrane D, Goebbels R M, Fdilat I, Guiot Y, and Gianello P, Impact of porcine islet size on cellular structure and engraftment after transplantation: adult versus young pigs, Pancreas 30: 138-147, (2005).
Hutton J C and Malaisse W J, Dynamics of O₂ consumption in rat pancreatic islets, Diabetologia 18: 395-405, (1980).
Kaihow T, Masuda T, Sasano N, and Takahashi T, The size and number of Langerhans islets correlated with their endocrine function: a morphometry on immunostained serial sectioned adult human pancreases, Tohoku J Exp Med 149: 1-10, (1986).
Kawashima Y, Yamamoto H, Takeuchi H and Kuno Y, Mucoadhesive DL-lactide/glycolide copolymer nanospheres coated with chitosan to improve oral delivery of elcatonin, Pharm Dev Technol 5:77-85 (2000).
Keegan M, Falcone J, Leung T and Saltzman W M, Biodegradable microspheres with enhanced capacity for covalently bound surface ligands, Macromol 37:9779-9784 (2004).
Krickhahn M, Buhler C, Meyer T, Thiede A, and Ulrichs K, The morphology of islets within the porcine donor pancreas determines the isolation result: successful isolation of pancreatic islets can now be achieved from young market pigs, Cell Transplant 11: 827-838, (2002).
Mattson G, Jansson L, and Carlsson P-O, Decreased vascular density in mouse pancreatic islets after tranplantation, Diabetes 51: 1362-1366, (2002).
Menger M D, Jaeger S, Walter P, Feifel G, Hammersen F, and Messmer K, Angiogenesis and hemodynamics of microvasculature of transplanted islets of Langerhans, Diabetes 38 Suppl: 199-201, (1989).
Petropavlovskaia M and Rosenberg L, Identification and characterization of small cells in the adult pancreas: potential progenitor cells?, Cell Tissue Res 310: 51-58, (2002).
Ritz-Laser B, Oberholzer J, Toso C, Brulhart M C, Zakrzewska K, Ris F, Bucher P, Morel P, and Philippe J, Molecular detection of circulating beta-cells after islet transplantation, Diabetes 51: 557-561, (2002).
Scharp D W, Lacy P E, Santiago J, McCullough C, Weide L G, Falqui L, Marchetti P, Gingerich R, Jaffe A, Cryer P, Anderson C, and Flye W, Insulin independence following islet transplantation into patient with Type I, insulin dependent diabetes mellitus, Diabetes 39: 515-518,(1990).
Shapiro A M, Lakey J R, Ryan E A, Korbutt G S, Toth E, Warnock G L, Knetman N M, and Rajotte R V, Islet transplantation in seven patients with type I diabetes mellitus using a glucocorticoid-free immunosuppressive regime, New England J Med 343: 230-238, (2000).
Sutherland D E, Gores P F, Farney A C, Wahoff D C, Matas A J, Dunn D L, Gruessner R W, and Najarian J S, Evolution of kidney, pancreas, and islet transplantation for with diabetes at the University of Minnesota, Am J Surg 166: 456-491, (1993).
Sweet I R, Khalil G, Wallen A R, Steedman M, Schenkman K A, Reems J A, Kahn S E, and Callis J B, Continuous measurement of oxygen consumption by pancreatic islets, Diabetes Technol Ther 4: 661-672, (2002).
Tatarkiewicz K, Garcia M, Lopez-Avalos M, Bonner-Weir S, and Weir G C, Porcine neonatal pancreatic cell clusters in tissue culture: benefits of serum and immobilization in alginate hydrogel, Transplanation 71: 1518-1526, (2001).
von Mach M A, Schlosser J, Weiland M, Feilen P J, Ringel M, Hengstler J G, Weilemann L S, Beyer J, Kann P, and Schneider S, Size of pancreatic islets of Langerhans: a key parameter for viability after cryopreservation, Acta Diabetol 40: 123-129, (2003).
Warnock G L, Ellis D K, Cattral M, Untch D, Kneteman N M, and Rajotte R V, Viable purified islets of Langerhans from collagenase-perfused human pancreas, Diabetes 38: 136-139 (1989).
Xu W, McDough R C, Langsdorf B, Demas J N, and DeGraff B A, Oxygen sensors based on luminescence quenching: interaction metal complexes with the polymer supports, Anal Chem 66: 4133-4141 (1994).

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. An implantable device comprising:
a substantially planar scaffold comprised of a biomaterial having a major surface; and
individual islet cells or small islet cell clusters attached to said surface of said biomaterial scaffold to form a multilayer of islet cells, said individual islet cells or small islet cell clusters being derived from adult intact islets.

2. The implantable device of claim 1 wherein said biomaterial scaffold is a flexible biomaterial.

3. The implantable device of claim 2 wherein said biomaterial is selected from the group consisting of poly(DL-lactide-co-glycolide) (PLG), polylactic acid (PLA), and poly(lactic-co-glycolic acid) (PLGA).

4. The implantable device of claim 1 wherein said multilayer comprises a combination of insulin-producing beta cells and other islet cell types.

5. The implantable device of claim 1 wherein said small islet cell clusters are derived from intact islets by culturing the intact islets in a calcium-depleted media.

6. The implantable device of claim 1 wherein said small islet cell clusters are derived from intact islets using enzymatic digestion.

7. The implantable device of claim 1 wherein said small islet cell clusters are derived from intact islets using a combination of culturing the intact islets in a calcium-depleted medium and enzymatic digestion.

8. The implantable device of claim 1 wherein said individual islet cells or small islet cell clusters form a multilayer of islet cells on said scaffold surface about 2 to 5 cells thick.

9. The implantable device of claim 1 wherein said individual islet cells or small islet cell clusters form a multilayer of islet cells on said scaffold surface about 10 to 50 microns thick.

10. The implantable device of claim 1 wherein said biomaterial has one or more cell adhesion molecules attached to the surface to facilitate attachment of individual islet cells or small islet cell clusters.

11. The implantable device of claim 10 wherein said cell adhesion molecules are selected from the group consisting of integrins, cadherins, selecting, and immunoglobulins.

12. The implantable device of claim 10 wherein said cell adhesion molecules are attached to the biomaterial scaffold surface by a covalent bond, and further comprising a spacer molecule between the cell adhesion molecule and the biomaterial scaffold.

13. The implantable device of claim 12 wherein said spacer molecule is about 11 to 46 angstroms.

14. The implantable device of claim 12 wherein the spacer comprises polyethylene, poly amino acids, or poly amino caproic acids.

15. The implantable device of claim 1 further comprising one or more angiogenesis factors, antibiotics, antioxidants, anti-cytokines, or anti-endotoxins controllably released from said scaffold.

16. The implantable device of claim 1 wherein said multilayer has a substantially uniform thickness such that the cell thickness varies by no more than 1-2 cells across the surface of the biomaterial scaffold.

17. A method for forming an implantable device comprising:
obtaining intact islets from a pancreas;
deriving individual islet cells or small islet cell clusters from said intact islets; and
attaching said individual islet cells and small islet cell clusters in a multilayer to a major surface of a substantially planar implantable biomaterial scaffold.

18. The method of claim 17 wherein said deriving step comprises subjecting the intact islets to enzymatic digestion, calcium depletion, or a combination thereof.

19. The method of claim 17 wherein said biomaterial is poly(DL-lactide-co-glycolide) (PLG), polylactic acid (PLA), or poly(lactic-co-glycolic acid) (PLGA).

20. The method of claim 17 wherein said multilayer is about 2 to 5 cells thick.

21. The method of claim 17 wherein said multilayer has a substantially uniform thickness such that the cell thickness varies by no more than 1-2 cells across the said surface of said biomaterial scaffold.

22. The method of claim 17 where said attachment step comprises centrifuging said scaffold while a first suspension of individual islet cells or small islet cell clusters in a liquid media is placed over said scaffold, thereby spinning said individual islet cells or small islet cell clusters onto said scaffold.

23. The method of claim 22 further comprising the steps of removing a portion of said liquid media from said first suspension, and then placing a second suspension of individual islet cells or small islet cell clusters in liquid media over said scaffold, and then centrifuging said scaffold again.

24. The method of claim 17 wherein said attachment step results in the formation of layers of cells on said scaffold about 10 to 50 microns thick.

25. The method of claim 17 further comprising the step of attaching one or more cell adhesion molecules to said scaffold to facilitate attachment of the individual islet cells or small islet cell clusters to said scaffold.

26. The method of claim 25 wherein said cell adhesion molecules are selected from the group consisting of integrins, cadherins, selecting, and immunoglobulins.

27. The method of claim 25 wherein said cell adhesion molecules are attached to the biomaterial scaffold by a covalent bond, and further comprising a spacer molecule between the cell adhesion molecule and the biomaterial scaffold.

28. The method of claim 17 wherein said small islet cell clusters are obtained from intact islets using a combination of culturing the intact islets in a calcium-depleted medium and enzymatic digestion.
